(19) United States
(12) Patent Application Publication
(10) Pub. No.: US 2016/0264687 A1
(43) Pub. Date: Sep. 15, 2016

(54) ELECTRON BEAM PRE-TREATMENT OF INORGANIC AND POLYMER SURFACES FOR BONDING OF DETECTABLE MARKER MOLECULES

(71) Applicant: APDN (B.V.I.) INC., Tortola (VG)

(72) Inventor: Phidung H. TRAN, East Setauket, NY (US)

(73) Assignee: APDN (B.V.I.) Inc., Tortola (VG)

(21) Appl. No.: 15/032,710

(22) PCT Filed: Oct. 31, 2014
(86) PCT No.: PCT/US14/63295

§ 371 Date: Apr. 28, 2016

Related U.S. Application Data
(60) Provisional application No. 61/899,099, filed on Nov. 1, 2013.

(51) Int. Cl. C08F 8/30; C12Q 1/68

(52) U.S. Cl. CPC C08F 8/30; C12Q 1/6876

(57) ABSTRACT

A method of binding a detectable marker to a surface comprising the steps of exposing the surface to an electron beam to produce an electron beam-treated surface; applying a detectable marker to the electron beam-treated surface; and thereby producing a surface-bound detectable marker on the treated surface. The detectable marker can be any suitable marker such as an optical marker, a dye, a fluorophore, a biomolecule, a metal, or a rare earth element. Also provided is a composition including a detectable marker immobilized on a surface pre-treated with an electron beam.

### TECHNICAL FIELD

The present invention relates to physicochemical treatments of surfaces and to methods of bonding marker molecules to such surfaces and subsequently detecting and/or extracting such marker molecules from such treated surfaces for authentication, tracking, and validation of the treated surface and for data storage of provenance information related to the particular object the surface of which is so treated.

### SUMMARY

In one embodiment, the invention provides a composition including a detectable marker immobilized on a surface pre-treated with an electron beam. The detectable marker can be any detectable marker, such as for instance, an optical marker, a dye, a fluorophore, a biomolecule, a metal or a rare earth element. In one embodiment, the detectable marker includes one or more nucleic acid molecules. In another embodiment, the composition the detectable marker is chemically bonded to the electron beam-treated surface. The surface pre-treated with an electron beam can be any surface, such as for instance an inorganic surface such as a metal, a ceramic, a semi-conductor, a crystal or a gemstone. Alternatively, the surface pre-treated with an electron beam can be a polymer and the polymer can be chemically bonded to one or more functional molecules, such as a stabilizer, a lubricant, a plasticizer or a fire retardant.

In another embodiment, the invention provides a method of binding a detectable marker to a surface, wherein the method includes the steps of exposing the surface to an electron beam to produce an electron beam-treated surface; and applying a detectable marker to the electron beam-treated surface to produce surface-bound detectable marker on the treated surface. The detectable marker can be any suitable detectable marker, such as for instance, an optical marker, a dye, a fluorophore, a biomolecule, a metal or a rare earth element.

In still another embodiment, the invention provides an authentication method including the steps of exposing a surface of an object, item or film to an electron beam to produce an electron beam-treated surface; and applying a detectable marker to the electron beam-treated surface producing a surface-bound detectable marker on the treated surface; extracting a sample of the detectable marker from the electron beam-treated surface; and identifying the detectable marker and thereby authenticating the object, item or film.

### BRIEF DESCRIPTION OF THE FIGURES

FIG. 1 illustrates the useful electron beam dose ranges—Rₒₚₜ is the Equal-entrance=Equal-exit depth depth-dose relations where Rₒₚₜ (optimum depth) is the equal-entrance, equal-exit depth parameter; R₅₀ is the depth where the exit dose is 50% of the maximum dose; R₅₀ₑ is the depth where the exit dose is 50% of the entrance dose; and Rₚ is the depth where the tangent line at the inflection point of the decreasing curve intersects the depth axis. Dₑ is the energy absorbed at the entrance level and D_{e/2} is half the Dₑ dose.

FIGS. 2A and 2B show a diagramatic comparison of the shallow penetration of ultraviolet light (FIG. 2A) versus the depth penetration of an electron beam (FIG. 2B) and crosslinking induced by each in a polymer film, such as a polyethylene film.

### DETAILED DESCRIPTION

As counterfeiters become more sophisticated, detection of counterfeit articles becomes more difficult and resource-intensive. One security approach is to provide the option for items to be marked in a manner that authenticates the item and may be used to verify the source of supply or to track an item in transit. The ability of custom markers to provide authentication has been successfully demonstrated when item marking is applied by the original equipment manufacturer (OEM), such as the security markers and marker systems offered by Applied DNA Sciences, Inc. (See www.adnas.com).

In an exemplary embodiment of the present invention the marker can be an optical marker. The optical marker can be any suitable optical marker, such as for instance, a fluorescent marker, an ultraviolet marker, an infrared marker, a dye such as a chromophore, a luminescent compound. In one embodiment, the optical marker is present in an ink.

In another embodiment, the invention provides a composition that includes a detectable marker immobilized on a surface that has been pre-treated with an electron beam. The detectable marker can be any suitable detectable marker, such as an optical marker, a dye, a fluorophore, a biomolecule, a metal or a rare earth element.

In one embodiment of the present invention, the marker molecule can be any suitable marker molecule, such as a biomolecule or an inorganic molecule. The biomolecule can be any biomolecule, such as for example and without limitation, a protein, a peptide, a nucleic acid, a protein-nucleic acid (PNA) complex, a carbohydrate, a fatty acid, a co-enzyme or a vitamin. The nucleic acid may include DNA or RNA, or a DNA:RNA hybrid. The nucleic acid may be single stranded or double-stranded, and may be composed of natural or non-natural sequences. See for instance US Pat. Nos. 8,124,33; 8,372,648; 8,415,164; 8,415,165; 8,420,400; and 8,426,216.

The inorganic molecule can be any inorganic molecule, such as for instance a metal, a non-metal or a rare earth element; or a salt or a chemical complex of the inorganic molecule. The metal can be any metal, such as for instance a transition metal, a lanthanoid, an actinoid, or an alkaline earth metal. The metal marker can be for instance and without limitation, an alkali metal such as magnesium, calcium, strontium, barium or radium. Alternatively, the metal can be a transition metal such as titanium, vanadium, chromium, manganese, iron, cobalt, nickel, copper, zinc, molybdenum, palladium, silver, cadmium, tungsten, platinum, gold and mercury. However suitable transition metals also include zirconium, niobium, technetium, osmium and iridium. The metal marker can be any rare earth element, such as for instance and without limitation, lanthanum, samarium, gadolinium, terbium, dysprosium, holmium or erbium.

Industrial metals useful in marker molecules of the present invention either separately or in mixtures or alloys of two or more, include iron (Fe), tin (Sn), lead (Pb), nickel (Ni), aluminum (Al), magnesium (Mg), titanium (Ti), tungsten (W), chromium (Cr), manganese (Mn) and molybdenum (Mo). The rarest metals by natural abundance include ruthenium (Ru), rhodium (Rh), palladium (Pd), tellurium (Te), rhenium (Re), osmium (Os), and iridium (Ir) are thus especially useful as markers. The precious metals platinum (Pt), gold (Au) and silver (Ag) can also be used in metal marker molecules in salts or chelates or as metallic elements.

The fluorophore marker can be any fluorophore, such as for instance a cyanine dye, a fluorescein dye, tetramethylrhodamine, Fluorescein, Fluorescein isothiocyanate (FITC), Dansyl, Texas Red, X NBD, NIR dye (near IR label with fluorescence emission wavelength greater than about 600 nm, such as carbocyanine dye (for example, an indocyanine dye), dye (e.g. Cy5, Cy5.5, and Cy7, each of which are available from GE Healthcare), Alexa dye (AlexaFluor dyes e.g. AlexaFluor660, AlexaFluor680, AlexaFluor700, AlexaFluor750 and AlexaFluor790 from Invitrogen), VivoTag-680, VivoTag-5680, VivoTag-5750, each of which are available from VisEn Medical; Tamra, (Dy677, Dy676, Dy682, Dy752, Dy780 from dyonics), DyLight547 and DyLight647, available from Pierce, HiLyte Fluor 647, HiLyte Fluor 680, and HiLyte Fluor 750, each of which are available from AnaSpec; IRDye 800, IRDye800CW, IRDye 800RS, and IRDye 700DX, each of which are available from Li-Cor; and ADS780WS, ADS830WS, and ADS832WS, each of which are available from American Dye Source. NIR labels can be enhanced by NIR fluorescence enhancement (NIR-FE). “NIR label” means a near-infrared label with fluorescence emission wavelength greater than about 600 nm, such as carbocyanine dye (for example, an indocyanine dye). Other marker dyes include 6-carboxyfluorescein (FAM), Redmond Red, Yakima Yellow, and Quasor670, although many others can be used (e.g., those disclosed in Haugland ed., The Handbook: A Guide to Fluorescent Probes and Labeling Technologies, 10th Ed., 2005, Invitrogen, Carlsbad, Calif.).

US2013/0172207 of Dai et al. entitled “Fluorescence Enhancing Plasmonic Nanoscopic Gold Films And Assays Based Thereon” discloses nanostructured gold films which can be produced by solution-phase depositions of gold ions onto a variety of surfaces. The resulting plasmonic gold films can be used for enhanced spectroscopic-based immunoassays in multiplexed microarray format with detection mechanisms based on either surface-enhanced Raman scattering or near-infrared fluorescence enhancement. The preparation of the films and subsequent modifications of the gold film surfaces provide increased sensitivity for detection purposes. The films are discontinuous, forming gold “islands.” Sensitivity, size, shape, and density of the nanoscopic gold islands comprising the discontinuous nanostructured gold film are controlled to enhance the intensity of Raman scattering and fluorescence in the near-infrared, allowing for improved measurements in clinical diagnostic or biomedical research applications.

In one embodiment, the detectable marker includes one or more nucleic acid molecules. In another embodiment, the composition the detectable marker is chemically bonded to the electron beam-treated surface. The surface pre-treated with an electron beam can be any surface, such as for instance an inorganic surface such as a metal, a ceramic, a semi-conductor, a crystal or a gemstone. Alternatively, the surface pre-treated with an electron beam can be a polymer and the polymer can be chemically bonded to one or more functional molecules, such as a stabilizer, a lubricant, a plasticizer or a fire retardant.

In another embodiment, the invention provides a method of binding a detectable marker to a surface, wherein the method includes the steps of: exposing the surface to an electron beam to produce an electron beam-treated surface; and applying a detectable marker to the electron beam-treated surface to produce surface-bound detectable marker on the treated surface. The detectable marker can be any suitable detectable marker, such as for instance, an optical marker, a dye, a fluorophore, a biomolecule, a metal or a rare earth element.

In still another embodiment, the invention provides an authentication method including the steps of: exposing a surface of an object, item or film to an electron beam to produce an electron beam-treated surface; and applying a detectable marker to the electron beam-treated surface producing a surface-bound detectable marker on the treated surface; extracting a sample of the detectable marker from the electron beam-treated surface; and identifying the detectable marker and thereby authenticating the object, item or film.

In one embodiment the surface that has been pre-treated with an electron beam for immobilization of a detectable marker can be a surface of a metal, a ceramic, a semi-conductor, a crystal or a gemstone.

In one embodiment the surface that has been pre-treated with an electron beam for immobilization of a detectable marker can be a metal surface, such as for instance a surface of an ingot, a bar, a shaped metal component, a beam, a wire, or a chain.

In another exemplary embodiment of the present invention the marker molecules of the present invention can be applied and bound to one or more surfaces of an object by any of a number of forces. The marker molecules can be bound to surfaces treated with an electron beam. For instance, the marker molecules can be bound to the electron beam-treated surface by weak forces (also known as weak atomic forces or Van der Waals forces), ionic interactions of cationic or anionic marker molecules with oppositely charged ions at the surface of the object to be marked, or by chemical bonding of the marker molecules through covalent bonds formed after exposure of the marker molecules to the electron beam-treated surface.

In another embodiment of the present invention the marker molecules can be entrapped in a matrix of an electron beam-treated polymer, wherein the polymer may comprise all or part of the object to be marked. In one embodiment the marker molecules can be entrapped in a matrix formed by cross-linking during electron beam treatment in the presence of the marker molecules. Alternatively, the marker molecules can be encapsulated in particles that may be applied to the electron beam-treated surface and bound thereto.

In one embodiment the bound marker molecules, polymer entrapped marker molecules or the encapsulated marker molecules can be released by treatment of the marked surface with a solvent. The solvent can be any suitable solvent that releases the marker molecule into solution. For instance, the solvent may be an aqueous or a non-aqueous solvent. The aqueous solvent can be water or any aqueous solution, such as a salt solution or a buffer solution.

In another embodiment of the present invention the marker molecules can be released by physical disruption of the marked surface, such as by scraping or shaving the surface to provide an amount of material sufficient to be analyzed for the presence of the marker molecule.

Electron beam (EB) accelerator devices used in industry for commercial activation of surfaces and for crosslinking of components of the exposed object, do not generate radioactivity and so do not face the same security, transportation and disposal issues as those of long lived, gamma-ray emitting isotopes, which are used in only a few industrial applications and for a few medical device sterilization applications.

Applications for industrial EB accelerators include, without limitation: the treatment of wire, cable and tubing; surface curing; shrink film wrapping and treatment of materials used in the manufacture of tires. High-current electron beam accelerators are widely used in many industries to alter the physical and chemical properties of materials and also for sterilization and detoxification. EB is also used for heat-shrinkable tubing and crosslinking polyethylene tubing. EB devices for surface curing are often mounted on printing presses and in coatings production lines. Most of the heat-shrinkable film as used for food packaging is crosslinked before the film is blown into its finished dimensions. The layers of automotive tires are crosslinked by EB irradiation.

Electron beam accelerators have the following characteristics: 1) electrons are emitted from heated cathodes; 2) electrons are focused into a beam with an extraction electrode; 3) electrons are accelerated within an evacuated space with a strong electric field. Electrons pass into the air through a thin metal-foil window usually of titanium. In microwave linear accelerators (linacs), the energy is determined by the electronic charge times the forward electric field integrated over the path length. The electron energy is expressed in electron volts (eV), kiloelectron volts (keV) or megaelectron volts (MeV).

Accelerated electrons and short wavelength X-radiation (4.1×10⁻³ nm) and gamma-ray (1.0×10⁻³ nm) photons interact with matter at the atomic level. Radio-frequency and microwave radiation, which can penetrate materials, require a polar material in order to induce sufficient molecular vibration to generate heat and thereby cause chemical or material responses. In industrial applications, light and ultraviolet (UV) radiation are limited to surface effects and depend upon the use of costly photo-sensitive compounds which decompose upon exposure and thereby initiate chemical reactions. The energy dependence on radiation wavelength is given by:

E=hc/λ

where E is the energy in joules; h is Planck's constant (6.626×10⁻³⁴ joule-seconds); c is the speed of light in vacuum in meters/second (2.998×10⁸ m/s) and λ, is the wavelength in meters. One electron volt (1.0 eV)=1.602×10⁻¹⁹ joules.

Two fundamental properties of all electron beam accelerators are the electron energy and the beam current. Electrons have mass and electrical charge, so that their penetration into materials is limited by their kinetic energy and by the mass and density of the target material. The amount of electron exposure, the absorbed dose, is measured in SI units as the gray, where 1 gray=10⁴ ergs/gram, or as commonly used in industrial processing, the kilogray (kGy) where 1 kGy=1 J/g absorbed energy per mass. The EB industry generally relies upon electron beam energies ranging from 75 keV to 10 MeV. Lower energies tend to lose excessive beam power in the beam window and in air; higher energies involve the risk of induced radioactivity. For mid-energy (500 keV to 5 MeV) and high-energy (5 MeV to 10 MeV) electron accelerators, it is common to express beam penetration on the basis of equal-entrance, equal-exit exposure in unit density material.

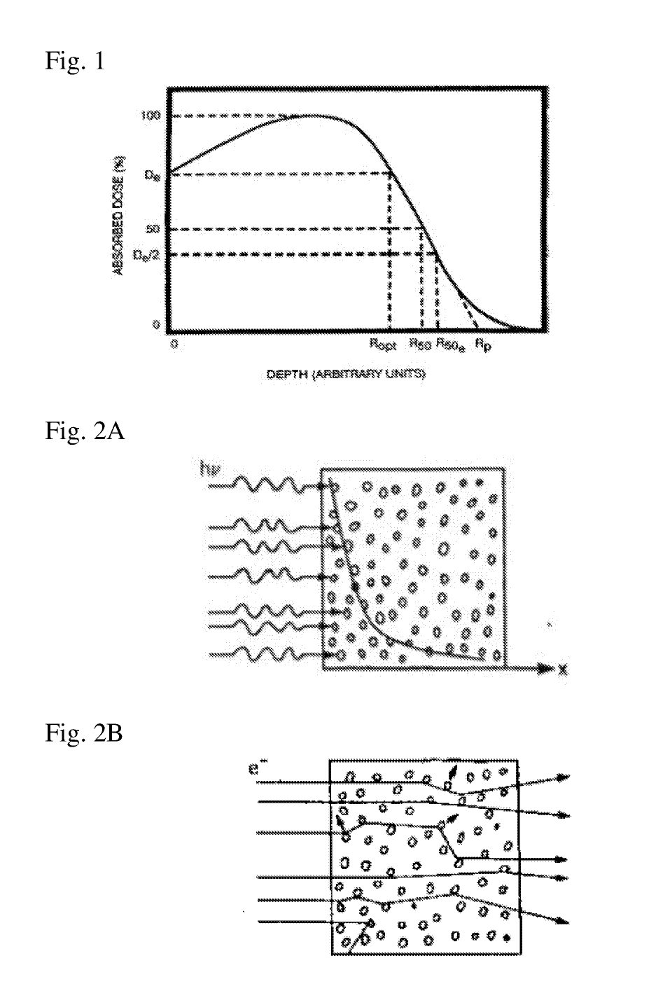

FIG. 1 shows a profile of dose vs. depth of penetration for an electron beam. As the power is increased from about 1.0 MeV to 5.0 MeV, the peak absorbed dose and Rₒₚₜ are shifted deeper into the material and Rₚ persists to a deeper level. Using the equal-entrance, equal exit criteria, the depth of penetration in microns (μm) is linearly related to the electron energy in keV.

Table I summarizes the electron energy ranges and typical penetrations for materials and products that are close to unit density and at commonly used electron beam energies.

TABLE I
Electron Beam Penetration - Market End-Uses

| | Electron | | |
|---|---|---|---|
| Market Segment | Energy | | Typical Penetration |
| Surface Curing | 80-300 | keV | 0.4 mm |
| Shrink Film | 300-800 | keV | 2 mm |
| Wire & Cable | 0.4-3 | MeV | 10 mm |
| Sterilization | 3-10 | MeV | 40 mm |

Corrections for material density can be made in order to assess the appropriate voltage for any particular application. For example, fillers used in coating formulations and in wire and cable compounds will increase product density. A carbon fiber composite (density 1.6 g/cm³) is only penetrated on an equal-entrance, equal-exit basis to 24 mm using a 10 MeV beam, whereas low bulk density items, such as packaged medical disposables (density 0.25 g/cm³), can be effectively penetrated at greater than 160 mm at 10 MeV. Due to the overlap of tail ends of the depth-dose penetration, opposite-sided electron beam exposure results in an effective 2.4 multiple of the EB penetration itself. For this reason, large, low bulk density packages can be effectively EB irradiated by turning over the item during processing.

High beam current distinguishes industrial electron beam accelerators from equipment that is commonly used solely for research purposes. Most industrial accelerators have beam currents in the tens of milliampere range (greater than 10 mA). Research equipment, such as Van de Graaff accelerators, Pelletrons™, and many linacs operate in the microamp range. High beam currents are desired in industry because product through-put rates are proportional to beam current. For estimating processing rates, an area through-put equation is often used.

Area processing rate=W_{b}V₁=6.0D(e)F(i)I/D

where Wb=beam width in m; V1=line speed in m/min; D(e) is the energy deposited per electron per areal density in units of MeV/(g/cm2); I=beam current in mA; D=dose in kGy; F(i) is the fraction of the emitted beam current intercepted by the irradiated material. In practice, the actors 6.0 D(e)F(i) are often represented by the letter “K” which is then called the Surface Area Rate or Processing Coefficient. The factor D(e) can be derived by an appropriate Monte Carlo code. The factor F(i) must be determined empirically based on the geometry of the irradiation process.

An equation derived from the area through-put equation is a product line speed equation wherein the factor “k” is the Area Processing Coefficient, K, divided by the web width, W. This is how the Linear Processing Coefficient, k, is commonly used in the low-energy EB area in relating beam current to line speed.

Line speed in meters/minute=k×beam current in mA/dose in kGy

wherein k is typically ˜10 to 30 depending on the electron energy, the web width, window thickness and air gap between the window and product.

TABLE II
Alternative Ionizing Radiation Sources

| | Electron Beams | X-rays | Gamma Rays |
|---|---|---|---|
| Power source: | Electricity | Electricity | Radioactive isotope |
| | | | (mainly cobalt-60) |
| Power activity: | Electrical on-off | Electrical on-off | 5.27 year half-life |
| Properties: | Electrons | Photons | Photons (1.25 MeV) |
| | mass = 9.1 × 10⁻³¹ kg | λ = 4.1 × 10⁻³ nm | λ = 1.0 × 10⁻³ nm |
| Charge: | 1.60 × 10−19 coulombs | None | None |
| Emission: | Unidirectional | Forward peaked | Isotropic |
| | (can be scanned and | | |
| | bent by magnets) | | |
| Penetration: | Finite range | Exponential | Exponential attenuation |
| | | attenuation | |
| Dose-rate: | 360,000 | 100 kilogray/hour | 10 kilogray/hour |
| | kilogray/hour | 2.7 × 10⁻² | 2.8 × 10⁻³ |
| | 100 kGy/second | kGy/second | kGy/second |

Industrial electron beam dose rates are in the order of 100 kGy/second or 360,000 kGy/hour. This is five orders of magnitude greater than the dose rates from cobalt-60 gamma-ray sources, which are about 10 kGy/hour or 2.8×10⁻³ kGy/second, as shown in Table II. Research accelerators, such as Van de Graaff generators, have dose rates of about 10 to about 100 kGy/hour, which is much closer to gamma-ray sources.

One useful electron beam device, the Dynamitron™, is an accelerator based on a parallel capacitive-coupled, cascaded rectifier, direct-current circuit. The Dynamitron™ operates at up to 5.0 MeV with total beam power up to 300 kW and attains the combination of higher electron energy and higher beam currents than many other contemporaneous systems.

### Industrial Electron Beam Sources

The major categories of electron beam devices based upon accelerator electron energy include. 1) high-energy units (5.0 to 10 MeV); 2) mid-energy, high-current units (400 keV to 5.0 MeV); and 3) low-energy, self-shielded units (80 to 300 keV).

### 2.3. High-Energy Accelerators

Two types of accelerator design have found industrial acceptance in the high energy area (5.0 to 10 MeV) area: microwave linear accelerators (linacs) and the radiofrequency Rhodotron. Linacs are used extensively in research and in medical therapy, but are less suitable for industrial use. For the most part, industrial linacs are limited to a peak energy of 10 MeV so as not to pose problems of induced radioactivity or used at reduced electron energies to avoid these issues.

Whereas industrially used linacs typically operated with a maximum of 60 kW of total beam power, the Rhodotron operates up to 700 kW at 7.0 MeV. At such high beam power, the generation of X-rays becomes a viable industrial option for radiation processing. The compact design of the Rhodotron provides the capability of multiple beam lines with different electron energies drawn from the same accelerator. Rhodotrons operate using magnets to accelerate electrons through a “figure eight” pattern.

Linacs have been used to produce X-rays and the Rhodotron has also shown industrial viability for X-ray processing. For example, the US Postal Service has been using a 130 kW Rhodotron installed in a facility in New Jersey to sanitize mail for critical US Federal government departments and agencies.

Examples of companies supplying the industrial market with high electron energy accelerators are listed below along with Ion Beam Applications, the manufacturer of the Rhodotron, an example of a high energy electron beam accelerator. Industrial linear accelerators (linacs) include:

Getinge Linac (formerly Linac Technologies) (www.linactechnologies.com);

Budker Institute of Nuclear Physics (www.inp.nsk.su);

L-3 Communications Pulse Sciences (www.titan-psd.com/TitanScan/index.html);

Mevex (www.mevex.com);

EB-Tech (www.eb-tech.com) with the Budker Institute of Nuclear Physics.

The Rhodotron:

Ion Beam Applications SA (www.iba.be/industrial/index.php).

### Mid-energy Accelerators

Mid-energy electron accelerators produce scanned beams that range in energy from 400 keV to 5 MeV. The units that are suitable for industrial use provide high beam currents of many tens of milliamps. High beam currents provide high dose-rates needed for industrial production. Five electrical design systems have been used to attain mid-energy and high beam current: 1) the Cockcroft-Walton and its enhancements by Nissin-High Voltage (developed by Mizusawa and associates), 2) the Insulated Core Transformer (developed by Van De Graaff, Trump and Emanuelson and enhanced by M. Letournel), 3) the Dynamitron (developed by Cleland and associates at RDI), 4) a magnetic coupled dc system (the ELV systems developed by Salimov at the Budker Institute), and 5) high-current pulsed beams (the ILU radiofrequency systems developed by Auslender and associates).

The Dynamitron can attain very high beam currents (60 mA) at up to 5.0 MeV (300 kW). The ICT and ELV mid-energy accelerator designs are limited in electron energy to 2.5 MeV.

Low-current mid energy electron accelerators have been developed for research functions and do not generally meet industrial high through-put requirements.

### Low-Energy Accelerators

Applications for low energy accelerators include the curing or crosslinking of inks, coatings and adhesives that are based upon liquid reactive materials that do not contain solvents. A driving factor has been the need to limit the air pollutants emitted from industrial operations. Low-energy EB processing have been found to have overall energy efficiency of these radiation processes far exceeding that of other methods, such as forced air drying. Low-energy accelerators are sufficiently low in voltage that they can be shielded with high density metal, most commonly lead, but more recently with steel. At the upper end of the low-energy systems are 500 keV scanned ICT accelerators that are used to crosslink films for food packaging with one end-user having >125 such EB units. Most low-energy electron beam accelerators are used printing, coating or similar continuous web-based processes.

Companies that supply self-shielded, high-current low-energy accelerators (<300 keV) include: Energy Sciences Incorporated (www.ebeam.com); Broadbeam Equipment (www.broadbeamequipment.com/home.shtml); NHV Corporation (www.nhv.jp/en/index.html); and Advanced Electron Beams (www.advancedelectronbeams.com).

Low-Energy EB Vs. Ultraviolet (UV) Radiation

An alternative process to low-energy EB for the curing of inks, coatings and adhesives, that also uses liquid applied materials with near-zero volatile organic compounds (VOCs), is ultraviolet (UV) radiation. UV sources tend to have peaked emissions, as between 240 nm and 270 nm and between 350 and 380 nm for mercury vapor sources. Metal dopants are used to shift the spectral output and interests have emerged in using light to initiate cure.

Electron beams penetrate through materials such that even very opaque coatings can be easily cured with EB. Since UV itself is too low in energy to initiate reactions, photoinitiators must be used in UV curable formulations. These specialty materials can add to the cost of a formulation and their absorption must be matched to a given UV source. These complex photochemical reactions can be up to 10 times slower than those initiated with EB. EB processes are more widely accepted where high volume, high speed production is required. See FIGS. 2A and 2B for a comparison of UV penetration versus EB penetration.

### Material Effects

Carbon based materials that are monomers, oligomers or polymers are used in industrial radiation processing. In dealing with polymers, the predominant chemical reaction of interest is the cleavage of carbon-hydrogen bonds to form free radicals, leaving atoms along a molecular chain with an unpaired electron. The EB treatment causes the formation of a carbon free radical from a carbon atom having six orbiting electrons picking up an additional electron. Free radicals can be either neutral or charged. The unpairing of electrons to form radicals can also result from electron removal. Carbon-halide and carbon-methide bond cleavage are also of industrial importance. The free radical opening of vinyl double bonds in elastomers is of significance in the crosslinking of tire components. The opening of terminal double bonds on monomers and oligomers is of importance in the crosslinking of polymeric precursors of materials used in inks, coatings and adhesives. These free radical reactions are initiated by direct impingement of a material by radiation, either in the form of accelerated electrons or the slightly less energetic X-rays. Such direct impingement of radiation on materials contrasts sharply with widely known and industrially used forms of energy transfer as convection heating and the use of thermo-chemical reactions, most of which require catalytic initiation. As pointed out, thermo-chemical systems are grossly inefficient in terms of energy transfer in comparison to radiation processes.

In some polymers, the formation of free radicals can alternately lead to scission or the breaking of the carbon based polymer chain. Controlling the scission of carbon polymers is of interest in some application areas. The cleavage of the double strands of deoxyribonucleic acid (DNA) is useful in the sterilization and decontamination processes. Another basic chemical response to radiation of industrial consequence is the trapping of energy from electrons or X-rays by cyclic ring structures such that the absorbed energy resonates within the carbon ring itself. Polymers based on ring structures, such as polystyrene (PS), polycarbonate (PC), and polyethylene terephthalate or polybutylene terephthalate (PET or PBT) are known for their radiation resistance, being able to be exposed to thousands of kGy with little effect on the material's mechanical properties.

Color-body formation is another chemical response to radiation used in industry, especially in the use of plastics exposed to radiation in sterilization processes. Medical devices themselves and their blister packaging materials are required to remain as near colorless as possible. Polyvinyl chloride (PVC) materials when exposed to radiation in the sterilization process should not turn dark brown. It is generally held that color-bodies are formed by non-carbon materials, the halide excitation in PVC, but also residuals in materials such as PC and PET. A material such as polysulfone (PSU), with its abundant internal sulfur linkages, has little chance of retaining its transparency and water-white color upon exposure to any form of radiation, becoming dark brown under exposures required for sterilization.

Cationic reactions have garnered some commercial interest in the radiation processing industry. These result from heterolytic bond cleavages that are generated from the dissociation of highly specialized initiation catalysts with this chemistry. With EB and X-ray initiation, only one initiation catalyst has been found to be effective, a sole-sourced iodonium salt. Cationic chemistry has been explored in the coatings industry and for use with matrix materials in prototype electron beam cured carbon fiber composites. The main advantage of this chemistry is that it is not inhibited by the presence of oxygen so that crosslinking can take place in air, not being perturbed by oxygen inhibition, a special concern in the coatings area. The catalysts used in this chemistry can be costly and have been found to lack long-term shelf-stability when used in formulated products. Cationic reactions are also sensitive to humidity and often require some thermal post-cure in order to go to completion.

The wide variation in polymer properties make polymers useful for many different applications. The demands of a given application may require a specific set of properties which in turn dictate the choice of polymer type and within a given type a specific grade. In general, one should be aware of molecular weight (weight average=Mw; number average=Mn), molecular weight distribution (Mw/Mn), and molecular weight between crosslinks (Mc). Molecular weight and molecular weight distribution influence how easily a high molecular weight polymer, either a thermoplastic or an elastomer, will process using melt processing equipment, such as extruders, molding presses and so forth. Mw is close to the viscosity average molecular weight, which for thermoplastics governs the melt flow of the material. For elastomers, low Mw also indicates ease of processing prior to crosslinking. Measurements for bulk melt or elevated temperature flow properties rely on different tests for different types of materials. For example, melt index (MI) is used for polyethylenes and melt flow rate (MFR) is used for polypropylenes; the lower the molecular weight, the higher the MI or MFR, when tested per ASTM D-1238, “Standard Test Method for Melt Flow Rates of Thermoplastics by Extrusion Plastometer”. For polycarbonates, ASTM D-3935 “Standard Specification for Polycarbonate (PC) Unfilled and Reinforced Material” is appropriate. For PET, intrinsic viscosity in solution is used, per ASTM D-4603 “Standard Test Method for Determining Inherent Viscosity of Poly (Ethylene Terephthalate) (PET) by Glass Capillary Viscometer”. For elastomers, the Mooney viscosity is most commonly used, ASTM D-1646 “Standard Test Methods for Rubber—Viscosity, Stress Relaxation, and Pre-Vulcanization Characteristics (Mooney Viscometer)”. Higher Mooney units indicate higher molecular weight for an elastomer. Vendor specifications include such industry recognized indicators of Mw. Reputable vendors will also be able to provide an indication of molecular weight distribution (MWD).

In industrial electron beam processing, crosslinking of materials is the dominant use of this process technology, in thermoplastics such as polyethylene, elastomers as used in tire components or polymeric precursors as used in inks, coatings and adhesives. Crosslinking is the formation of a three-dimensional polymer network (ASTM D-883, Standard Definitions of Terms Relating to Plastics). Such networks are insoluble, forming a gel in solvents. The linking of molecules without gel formation is considered to be chain extension which results in increases in molecular weight (Mw) and shifts in molecular weight distribution (MWD). The morphology or structure of any given material governs many mechanical properties and physical attributes of a crosslinked product. However, in many applications the crosslinked density, reflected in the molecular weight between crosslinks, Mc is important.

In using radiation processing in the production of grafted polyethylene films for battery separators, a very high crosslinked density is desired to control ion flow. In tire manufacture, low crosslinked density and modest gel content (˜30 to 60%) is desired in order to permit the tire components to knit or flow together in subsequent molding operations which rely upon thermal curing. In heat-shrinkable products, films or tubings, Mc governs the elastic recovery force when a crosslinked product is taken above its melt transition to shrink around an object.

In coatings, controlled Mc is needed in order to balance surface hardness and impact resistance. In pressure sensitive adhesives (PSAs) that are crosslinked using electron beams, a controlled Mc is needed to balance tack and hold properties.

The three fundamental responses of polymeric materials to electron beam or X-radiation are: Crosslinking—the formation of an insoluble material; Scissioning—the lowering of the molecular weight of a material; or Neutral or little to no effects on mechanical properties. Polymers useful for coatings, films and adhesives that can be EB-treated to increase crosslinking and provide new properties include polyethylene (PE), coplolymers such as polyethylene and ethylene-propylene (EPM) copolymers, or polyethylene and ethylene-propylene-diene (EPDM) elastomers for flexibility.

### Polyethylenes

There are four major market end uses that rely on the radiation processing of polyethylenes (PE): 1) wire and cable insulation, 2) heat-shrinkable tubing and wraps, 3) heat-shrinkable food packaging films, and 4) controlled density closed-cell foams.

More than half of all of the industrial electron beam accelerators are used in crosslinking polyethylene. The radiation responses of various polyethylene types have been characterized by major users and some suppliers. In general, Gx is ˜1 and Gs<0.1. High density polyethylene (HDPE) with its higher crystallinity requires fewer crosslinks for property enhancement and thus often less EB exposure than lower density polyethylenes. Linear low density (LLDPE) polyethylenes with a broad molecular weight distribution (MWD) have been reported to have better response to radiation than the more common narrow MWD LLDPEs. The relatively new metallocene catalyzed polyethylenes (mPE) respond positively to radiation and crosslink well. The influence of comonomer type and content also affects the relative exposure needed for crosslinking. Copolymers with acrylate comonomers (methyl or ethyl, EMA or EEA) respond better than more common vinyl acetate comonomers (EVA) at the same comonomer content. Since comonomer content detracts from some of the desirable properties of polyethylenes, such as resistance to moisture vapor (ASTM F-1249 “Standard Test Method for Water Vapor Transmission Rate Through Plastic Film and Sheeting Using a Modulated Infrared Sensor” MVTR) and good dielectric properties, it will be interesting to see if blends with mPEs with their outstanding optical clarity and non-polar comonomers can replace more traditionally used copolymers. Blends with mPE will effectively lower combined melt transitions, but not detract from moisture barrier or dielectric properties. They can also impart the suppleness and impact resistance traditionally found with copolymers.

Ethylene copolymer made with vinyl alcohol, ethylene vinyl alcohol (EVOH), responds positively to radiation and is a preferred gas barrier inter-layer in food packaging films that are radiation crosslinked to make heat shrink films or as films for pre-packaged foods that will be irradiated. Ultra-high molecular weight polyethylenes (UHMWPE) respond well to electron beam radiation.

PE crosslinking, that is the formation of a three-dimensional insoluble polymer network, proceeds via the abstraction of a hydrogen atom from the saturated PE backbone. The free radical left on the carbon chain then finds another free radical site on an adjacent carbon on another molecule to form a crosslink, with the abstracted hydrogen combining with another abstracted hydrogen to form a gaseous, readily diffused by-product, molecular hydrogen (H₂).

### Polypropylenes

Polypropylene (PP) is used in medical device manufacture because of its stiffness and greater resistance to thermal distortion (Td) than that of even the highest density (0.965) HDPE, about 20° C. greater in standard heat deflection temperature tests, such as ASTM D-648. However, when exposed to radiation, polypropylenes are known to chain scission. This issue, which is aggravated by the presence of oxygen, has been successfully overcome. A long-lived trapped methide radical, CH3*, was identified as the source of continued polymer degradation. Stabilizer systems were developed that quenched this long lived radical and inhibited oxidative degradation as well. These systems enabled medical products, such as syringes, to be sterilized using radiation processing, notably electron beam sterilization.

The recently developed metallocene polypropylenes (mPP) exhibit the same degradation responses as more crystalline polypropylenes. The suppleness of the mPP resins indicates they may be more suited for packaging films than for device manufacture. Depending upon the test protocol one uses to evaluate radiation stability, the very low modulus of mPP can confuse the interpretation of results. Because of their excellent optical clarity, mPP as well as mPE have been proposed for use in blends with polypropylene (PP) as an alternative to plasticized polyvinyl chloride (PVC).

One problem overcome by appropriate reformulation was that upon exposure to ionizing radiation, polypropylene formed a methide radical which remained trapped within the crystalline domains of the polymer. Over time this trapped radical, especially in the presence of oxygen, would propagate chain scissioning and embrittlement of the base polypropylene grade.

The formulation of polypropylene copolymers containing small amounts of ethylene, produces different grades that have been shown to crosslink upon exposure to an electron beam. This permits the use of polypropylene in market areas such as wire and cable jacketing and closed cell foam. The key is the use of multifunctional monomers, such as trimethylol propane triacrylate (TMPTA) or triallyl cyanurate (TAC), to shift the kinetics of the free radical reaction from scissioning to crosslinking. These same additives are also known to accelerate the radiation response of polyethylenes. Studies have shown that properly formulated radiation tolerant polypropylene exhibits almost the same response, lack of embrittlement, when exposed to X-rays as it does when exposed to electron beams at a prescribed sterilization dose of 25 kGy, but suffers greater degradation when exposed to comparable doses of low dose-rate gamma-ray radiation. Table III below summarizes typical results.

TABLE III
Radiation Tolerant Formulated Polypropylene Copolymer

| Source | Dose | Dose-rate | | Elongation | Response |
|---|---|---|---|---|---|
| Control | None | — | | 100% | — |
| Gamma | 25 kGy | 2.8 × 10⁻³ | kGy/s | 30% | Scissioned |
| Gamma | 50 kGy | 2.8 × 10⁻³ | kGy/s | 2% | Scissioned |
| X-ray | 25 kGy | 3.3 × 10⁻² | kGy/s | 420% | Crosslinked |
| X-ray | 50 kGy | 3.3 × 10⁻² | kGy/s | 20% | Scissioned |
| EB 2 | 5 kGy | 100 | kGy/s | 410% | Crosslinked |
| EB | 50 kGy | 100 | kGy/s | 500% | Crosslinked |

### Halogenated Plastics: Polyvinyl and Polyvinylidene Chloride, Fluoropolymers

Polyvinyl chloride (PVC) is used in wire and cable jacketing because of its inherent flame retardant nature and its relatively low cost. PVC itself will degrade when exposed to radiation. However, it has long been known that multifunctional monomeric additives prevent this chain scission and enable PVC crosslinking during electron beam irradiation. In the medical device area, discoloration of PVC materials, such as tubing, bags and other low cost medical supply items when exposed to radiation sterilization is a disadvantage. This issue has been overcome by the use of additives. Without appropriate formulating, PVC will undergo chain scission upon exposure to ionizing radiation and yield corrosive decomposition products.

Another chlorinated thermoplastic material, polyvinylidene chloride (PVdC) is known for its gas barrier properties, as in the commercial film Saran Wrap™, however, this polymer not only severely discolors but also undergoes chain scissions upon exposure to electron beam or gamma-ray radiation. For these reasons PVdC is not useful as an inner-layer in irradiated food packaging films.

In general, polymers with tetra-substituted carbon atoms tend to degrade and undergo chain scission on EB exposure. This is the case for PVdC, for polyiso-butylene (PIB) and its isoprene copolymers, butyl rubbers, for polymethylmethacrylate (PMMA) and for polytetrafluoroethylene (PTFE).

Polyvinylidene fluoride (PVdF) is an exception. PVdF radiation crosslinks and is useful as a high temperature jacketing on wire, especially aircraft wiring, and for certain specialty heat shrink products. Fluoropolymers are useful for high temperature applications, due to their heat resilience and chemical and oil resistance and for their flame retardant properties. An alternating copolymer of ethylene and tetrafluoro-ethylene (ETFE) is also radiation crosslinkable and due to its elastomer properties is also useful for wire and cable jacketing. A copolymer of hexafluoropropylene and tetrafluoroethylene (FEP) will undergo chain scission when irradiated at room temperature, but can be crosslinked if the radiation is conducted slightly above its melt transition, i.e. above 260° C. Similar results have been found for PTFE when electron beam exposure is conducted above the melt transition (340° C.) and in an inert atmosphere. Crosslinking takes place and upon cooling, PTFE loses its highly crystalline structure and a near-transparent material results. The commercial merits of high temperature FEP or PTFE crosslinking should be weighed against the performance properties obtained with more easily processable fluoropolymers, as PVdF and ETFE, which will crosslink at room temperature on exposure to radiation.

With irradiated thermoplastics such as polyethylene (PE) and polypropylene (PP) and with the fluoro-polymers (FPs), crosslinking and scissioning take place in the amorphous regions of the polymer. Crystalline domains which determine melt transitions, Tm, are thus mostly unaffected. The properties of a thermoplastic above its melt transition can be improved by electron beam crosslinking. Thermal mechanical analysis (TMA), as in ASTM E-1545, or differential scanning calorimetry (DSC), as in ASTM E-1356, can also be used to determine melt transitions and will still show melt transitions even as the crosslinked polymer is heated in a test instrument. Since the radiation response of polymers is grade specific, one cannot simply rely upon general material properties to determine a suitable grade for use in a material that will undergo radiation crosslinking. Commercial polymers are usually manufactured with stabilizers needed to protect the polymer from thermal conditions encountered during their manufacture. Such stabilizers can affect radiation response, as can slight changes in polymer morphology.

### Engineering Thermoplastics

Plastics such as the radiation tolerant polystyrene (PS) and polyethylene terephthalate (PET) and the clear, but chain scissioning-prone polymethylmethacrylate (PMMA) have been considered for use in medical devices and in the rigid clear blister packaging used for devices. These plastics have comparatively low heat distortion temperatures, Td, as determined by ASTM D-648, Standard Test Method for Deflection Temperature of Plastics Under Flexural Load in the Edgewise Position. For PS, Td is about 75° C. to about 95° C.; for PET about 70° C. and for PMMA about 80° C. to about 105° C. For medical devices that must not only tolerate radiation sterilization during production processes, but may be required to tolerate subsequent steam sterilization, polycarbonate (PC) with a much higher heat distortion temperature, Td about 140° C., is preferred. Major suppliers of PET have worked on retaining the optical clarity and water-white color of this resin when exposed to the demands of radiation sterilization.

Table IV summarizes the radiation response for these various plastic materials.

TABLE IV
Properties of Plastic Polymers

| | Thermal Properties, | | | |
|---|---|---|---|---|
| | ° C. | | | Radiation |
| Polymer | Tm | Td | Density | Response |
| Polyethylenes: | | | | |
| metallocene (mPE) | 60-105 | — | 0.870-0.915 | X |
| low density (LDPE) | 98-115 | 40-44 | 0.917-0.932 | X |
| linear low density (LLDPE) | 122-128 | 55-62 | 0.918-0.940 | X |
| high density (HDPE) | 130-137 | 79-91 | 0.952-0.965 | X |
| ultrahigh mol. weight (UHMWPE) | 125-135 | 68-82 | 0.940 | X |
| vinyl acetate copolymers (EVA) | 61-105 | — | 0.925-0.960 | X |
| acrylic acid copolymers (EAA | 94-102 | — | 0.924-0.958 | X |
| methyl acrylate copolymers (EMA) | 75-102 | — | 0.928-0.945 | X |
| ethyl acrylate copolymers (EEA) | 95-98 | 31-33 | 0.930-0.931 | X |
| butyl acrylate copolymers (EBA) | 86-93 | — | 0.926-0.928 | X |
| vinyl alcohol copolymers (EVOH) | 156-191 | 80-100 | 1.120-1.200 | X |
| Polypropylenes: | | | | |
| metallocene (mPP) | 149 | 94 | 0.900 | S |
| homopolymer (PP) | 168-175 | 107-121 | 0.900-0.910 | S |
| ethylene copolymers (PP) | 131-164 | 71-115 | 0.890-0.910 | S/X |
| Halogenated polymers: | | | | |
| unplasticized vinyl Cl (PVC) | 75-105 | 57-82 | 1.300-1.580 | S/X |
| vinylidene chloride (PVdC) | 150 | — | 1.600-1.780 | S |
| vinylidene fluoride (PVdF) | 135-175 | 68-140 | 1.760-1.800 | X |
| ethylene-tetrafluoroethylene (ETFE) | 270 | 81 | 1.700-1.720 | X |
| fluoroethylene-propylene (FEP) | 257-263 | 70-77 | 2.130-2.150 | S |
| ethylenechlorotrifluoroethylene | 220-240 | 90-92 | 1.680 | S |
| (ECTFE) | | | | |
| tetrafluoroethylene (PTFE) | 230 | 73-140 | 2.150-2.300 | S |

Suppliers of polycarbonates have also developed grades that do not discolor upon irradiation. This engineering thermoplastic can be used in producing devices, such as dialysis filter cartridges and other formed and molded articles. Because of their ring structures, PS, PET and PC are inherently radiation resistant. The issue of the retention of optical properties during radiation processing has been resolved for PET and PC, with radiation tolerant PET exhibiting less discoloration.

### Rigid Clear Plastics:

| | | | | |
|---|---|---|---|---|
| polystyrene (PS) | 83-100 | 78-103 | 1.040-1.080 | O |
| polymethylmethacrylate (PMMA) | 100-105 | 80-103 | 1.150-1.190 | S |
| polyethylene terephthalate (PET) | 243-250 | 68-72 | 1.300-1.330 | O |
| polycarbonate (PC) | 143-150 | 115-143 | 1.170-1.450 | O |

X = crosslinks;
S = scissions;
S/X = scissions, formulations crosslink;
O = neutral.

For PS, PMMA and PC, the glass transition temperature, Tg, is listed and not the melt transition, Tm; Td=the heat deflection temperature per ASTM D-648 at 0.46 MPa. For PS, PMMA and PC, the glass transition temperature, Tg, is listed and not the melt transition, Tm; Td=the heat deflection temperature per ASTM D-648 at 0.46 MPa.

### Elastomers

Unlike thermoplastics, whose dominant market uses rely upon properties attainable as non-crosslinked materials, elastomers require crosslinking in order to exhibit commercially useful properties. Commonly used elastomers have a reactive double bond within their polymer structure. Radiation exposure opens these double bonds to form crosslinks. Radiation crosslinking of natural rubber was one of the first industrially significant discoveries involving electron beam effects on materials. The polymers commonly used in tire manufacture are radiation crosslinkable: cis-polybutadiene (BR) for long wearing tread compounds, natural rubber (NR) or synthetic polyisoprene (IR) and styrene-butadiene (SBR) for blends and ethylene-propylene diene rubbers (EPDM) for ozone resistant sidewalls. The response of each elastomer type is dependent not only on specific characteristics of the elastomer but also upon the formulation. Even a polymer type known to undergo chain scission with radiation, the isobutylene-isoprene copolymer butyl rubber (IIR), when halogenated (as bromobutyl rubber—BIIR) and properly formulated can crosslink under radiation and is suitable for use in tire inner liners. Halogenated isobutylene-isoprene copolymers can be dehydrohalogenated to yield a conjugated diene butyl (CDB), which will undergo radiation crosslinking even without formulation.

### Thermoplastic Elastomers

Thermoplastic elastomers (TPE) can be either block copolymers or blends of a thermoplastic polymer and an elastomer. Block copolymers are reactor produced materials which can be processed as thermoplastics, but when cooled exhibit rubbery or elastomeric properties. These are styrene-isoprene-styrene (SIS), styrene-butadiene-styrene (SBS) or the saturated mid-block materials, styrene-ethylene/butylene-styrene (SEBS) or styrene ethylene/propylene-styrene (SEPS) copolymers. By reducing the styrene in SIS or using a saturated mid-block copolymer, these materials can be formulated to be EB-responsive.

Thermoplastic elastomers based on polyolefins (TPO) are blends of PEs or PPs with EPDM elastomers wherein the elastomer is often crosslinked using thermo-chemical systems. TPOs more suitable for medical products which produce no chemical residuals and can be made using EB processing to crosslink the elastomer portion in elastomer-plastic blends. The thermoplastic governs the melt transition and thus the extrusion properties of TPOs. The radiation response of these materials is also governed by the choice of the thermoplastic.

### Monomers and Oligomers

Monomers and low molecular weight oligomers, typically Mw<40,000, are radiation polymerized in-situ to form the crosslinked binders of inks and of coatings and to form functional adhesives. Low-voltage electron beams are used with these materials in high volume operations. These materials are formulated and then applied as liquids. Radiation crosslinking yields functional materials with there being little to no emission of volatile organic compounds, (i.e. near-zero VOCs). As a result, this technology has gained recognition as an inherently “green” chemistry. Free radical in-situ polymerization and crosslinking often relies upon the terminal vinyl or unsaturation of acrylate monomers. Monofunctional, di-functional and poly-functional monomers are typically very low in viscosity. Specific monomers are chosen for defined end-use performance properties. Monofunctional monomers are either acrylate or methacrylate materials with differing substitutive groups (R) that polymerize via their double bonds with themselves and with other monomers or oligomers into a crosslinked network. Di-functional monomers also have various mid-section constituent groups, but are also terminated with an acrylate double bond. A commonly used tri-functional acrylate is trimethylol propane triacrylate (TMPTA). This is also used to enhance the radiation response of polyethylenes and polypropylenes, as in wire and cable formulations and radiation crosslinkable polypropylenes.

To attain properties such as elongation or flexibility, low viscosity radiation curable formulations often also contain oligomers of higher molecular weight that are a viscous liquids. The substitutive R group in oligomers can be an epoxy, a polyester, a urethane or even an acrylate structure. The resulting crosslinked system then exhibits more of the properties of this substituent. Commonly used acrylated oligomers are liquid materials that have molecular weights (Mn) of less than 10,000 Daltons.

### Water Soluble Polymers

Water soluble polymers such as polyethylene oxide (PEO), and polyvinylpyrrolidone (PVP) are used in the radiation formation of hydrogels. Solutions of only about 4% to about 10% of PEO in water will form a gel at very low doses, typically less than 10 kGy. PVP is also very radiation responsive. Polyethylene glycols (PEG) and polyvinyl alcohols (PVA) have also been used in these systems, but PEO is the most widely used. The ethylenic structure of this polymer is very amenable to radiation crosslinking.

### Grafting

Radiation grafting is a useful technique for modifying the surfaces of materials. Grafting adds a monomeric or low molecular weight moiety to a high molecular weight formed polymer, which can be a film, a non-woven, a microporous film or a bulk material, to affect various properties. Grafting is defined as the ability to attach or grow a different material onto the backbone of another. With polymeric materials, the “different” material is usually a monomer and the “backbone” is a polymer or other solid. A chemical bond is then formed between the grafted moiety and the material. Low-energy EB processing is especially suitable for surface grafting.

Grafting has been used to enhance the biocompatibility of polymers. Grafting is used to control the ion flow through battery separators, the hydrophilic or hydrophobic properties of filters, and is useful for fuel cell membranes. Silanes can be grafted onto polymer film surfaces to impart release or non-adherent properties. Table V lists the variety of uses and potential applications for the EB grafting of materials.

TABLE V
EB Grafting of Polymers

```
Accepted commercial uses:
      1 - Battery separator membranes;
      2 - Micro-porous membranes and non-wovens;
      2 - Release coated films and papers;
Developed uses in early stages of commercialization:
      1 - Absorbents for metal ions;
      2 - Odor absorbent fabrics;
      3 - Substrates for cell tissue growth;
      4 - Surface modification of glass windows for ease of cleaning;
Developmental uses:
      1 - Compatibilization in heterogeneous composites;
      2 - Fluoropolymer membranes for fuel cells;
Long range opportunities:
      1 - Select grafted films for biomedical use as transdermal systems;
      2 - Modification of fabrics for flame retardancy;
Known uses not commercialized:
      1 - Bulk polymer modification to enhance adhesion;
      2 - Ion exchange membranes;
      3 - Controlled gas permeation of food packaging films.
```

### Natural Polymers

Cellulose is known to undergo chain scission when exposed to irradiation. With low energy EBs, the doses needed to cure inks or coatings are generally low and beam penetration is limited so that possible degradation of paper substrates is minimized. Higher doses can cause darkening or even singing of paper.

### End-Use Applications

### Wire and Cable Insulation

The electron beam crosslinking of the insulation jacketing on wire and cable is one of the most well established industrial uses of EB processing. EB treatment of wire and cable polymer jackets are inexpensive to process in production of the wire and cables and have significant advantages over chemical crosslinking. The ionizing energy provided by EB can be made efficient in crosslinking, optionally with the use of specific crosslinking activators to render the process even more efficient, without the heating or extended curing times generally necessary for chemical crosslinking. Electron beam-mediated crosslinked jackets for wires and cables have several additional useful properties: they are abrasion resistant and do not melt or flow at elevated temperatures such as when heated by electrical short circuits; they are not flammable even when exposed to soldering temperatures in circuit building; and they do not melt flame when installed near hot engine parts such as exhaust systems in cars, busses and other vehicles.

Polyvinyl chloride (PVC) and polyethylene (PE) coatings and covering are widely used for low voltage wires and have advantages of cost and speed of manufacture over continupous vulcanization processes. The PVC can be used to coat wires with braided cotton, fire retardants or lacquers or other insulating covering layers. Tetraethyleneglycol dimethacrylate (TEGDM) can be added to the PVC to increase crosslinking during EB exposure and stabilizers and plasticizers can also be added, as well as lubricants where relevant to smoothing the production process or the end use of the wire or cable.

Low smoke and low toxicity jacketing that is flame retardant and halogen-free can be made for instance from ethylene vinylacetate (EVA) and aluminum trihydrate (ATH). Aircraft standards for flame retardant jacketing are even more restrictive, and polyfluoropolymers such as ethylene tetrafluoro-ethylene (ETFE) are used in thinner coatings that are lighter and can withstand exposure to a 300 degree Celsius temperature range.

Crosslinking prevents insulation from dripping off an over-heated wire, as could result from a short circuit, or when exposed to the high heat of an automotive engine or even a fire. Specialized under-beam fixtures have been developed to transport wire using multiple passes under the beam. The wire is slightly turned during each pass so as to improve the uniformity of exposure even if the copper conductor would be thick enough to prevent beam penetration. Cross-firing beams at plus and minus 45 degrees can also enhance dose uniformity. Pay-off and take-up equipment has been designed so that the entire process can run at several hundred meters per minute. Depending upon the end-use requirement, wire jacketing is most often made from formulated polyethylene. Blends of polyethylene and ethylene-propylene rubber are used if greater flexibility is needed, especially as the diameter of the jacketing increases as for cables. When enhanced temperature resistance is required, polyvinylidene fluoride or other fluoropolymers are used. Fluoropolymers have the advantage of being oil resistant and flame retardant, but are also more expensive base materials.

A Typical Flame Retardant Wire and Cable Formulation includes 100 parts by weight PE/EPDM; 250 parts by weight of Hydral 710; parts by weight; 5 parts by weight of Zinc oxide; 10 parts by weight of a processing aid; 2 parts by weight of Silane A-172; 1 part by weight of an antioxidant; and 5 parts by weight of TMPTA/TAC.

The appropriate PE and/or EPDM formulation depends upon a variety of end-use and process considerations. Hydral is aluminum tri-hydrate and is a preferred flame retardant that liberates its water of hydration when exposed to flames in contrast to chlorinated materials which give off toxic gases as by-products. The process aid additive is typically an oil that enhances the ability to extrude such compositions. The silane additive is a coupling agent that improves the inter-action between the polymers and the aluminum tri-hydrate. Trimethylol-propane triacrylate (TMPTA) enhances the radiation response as does tri-allyl cyanurate (TAC). Crosslinking imparts two main properties to wire jacketing. First, should the wire itself become heated, for example, due to an electrical short circuit, the crosslinked jacketing will not melt or drip from the wire and will maintain its insulation. This is very important for under-the-hood wires used in automobiles.

### Heat-Shrinkable Tubing

Heat-shrinkable tubing is first extruded and then irradiated to a specified exposure as for insulated wire. The crosslinked tubing is then expanded. During the expansion process the tubing is heated in a chamber to above the melt transition of the plastic, typically polyethylene. Since the plastic is crosslinked, it behaves as a weak rubber and can be stretched or expanded by controlled air pressure differentials inside and outside the tubing. The stretched tubing is then quickly cooled so as to lower its temperature below what would have been its melt transition temperature. The crystalline domains in the polymer then form again and serve to hold the plastic in the stretched or expanded state. Pieces of tubing are then used to cover wire connections with the tubing having many of the same properties as the wire jacketing. An adhesive or sealant can be coated within the tubing so that a waterproof seal is made over any connection. Upon being heated during application, the tubing contracts and conforms to the connector or object inside it.

Heat recoverable closures for telecommunication splices and wraps for welded pipe joints have also been made. A heat recoverable sheet supplied as 35 cm wide tape was used as the supplementary corrosion protection for the 600 kilometers of the below grade sections of the 120 cm diameter Alyeska pipeline. This material has been shown to remain functional after over 30 years of service in harsh conditions.

### Heat-Shrinkable Food Packaging Films

An irradiation process for producing heat-shrinkable films for food packaging includes spreading an extruded tubular form under a beam, thereby absorbing most of the beam output, and then blowing the irradiated material into the final film dimensions. This became known as the “double-bubble” process—one bubble being the extrudate and the other the blown film. Heat-shrinkable films can consist of multi-layer co-extrusions which have five or more layers: 1) a direct food contact layer of PE; 2) a tie-coat layer of a polyethylene copolymer; 3) a gas barrier layer, typically made from EVOH; 4) another tie-coat layer; and 5) an exterior layer PE for abrasion resistance on which information can be printed. Heat-shrinkable food packaging films can be made using low-energy (300 keV) EB units with flat extruded sheets. The sheet is extruded and then irradiated and finally stretched using a tenter as commonly used in the plastic film industry to orient films. Self-shielded, 500 keV accelerators are preferred for this process.

### Closed Cell Polyethylene Foams

Since electron beam processing takes place at ambient temperatures, PE to be blown can be crosslinked without inadvertently activating a blowing agent. Radiation crosslinking eliminates the attempt to use two competing thermo-chemical reactions, one to crosslink the PE and another to blow it into foam. With radiation processing, extruded PE with a blowing agent incorporated in it is crosslinked under an electron beam with minimal or no thermal input. The extrudate is then brought between plates and heated to release the gas from the blowing agent. The type of PE used, the amount of blowing agent, the radiation exposure and process for blowing, all contribute to a well defined closed cell foam structure. A myriad of uses for these radiation crosslinked PE foams, including significant uses in automobiles for safety and protection, most notably as a cushioning under the interior header. Such foams are also used as backing materials in the medical device industry.

### Tire Components

The ability to tightly control electron beam exposure enables tire manufacturers to only partially cure or crosslink elastomers. Elastomer components are extruded and then irradiated to bring them to a gel or green state. In that gelled state, the elastomers are tougher than the non-cured materials and this prevents tire cord distortion or strike-through during subsequent molding operations. The finished tire is knitted or fused together during the final thermal molding process. Different elastomers are used for different functions in a tire. Properly formulated halogenated butyl rubbers (as BIIR) are used for innerliners. Sidewalls are made from ethylene-propylene rubbers (EPDM) because of the inherent ozone resistance of this polymer. Chafer strips are also partially EB cured.

### Inks, Coatings and Adhesives

Curable coatings have found major market uses on a variety of substrates, such as paper, wood, metals and plastics. An advantage to EB curing and crosslinking of coatings is that pigmentation does not interfere with the crosslinking process as it does with the use of ultraviolet radiation. Likewise, metallic pigments can be used.

Electron beams are more often used with wideweb presses for high volume production and for printed items that require outstanding graphics and color highlights. Electrons have the ability to penetrate pigments, whereas UV does not. EB ink formulations tend to be considerably less complex than UV formulations. Because electron beam processing is not a thermal means of energy transfer and takes place at near ambient temperatures, EB “drying” of inks can be used on heat sensitive substrates, such as plastic films, minimizing concerns over film distortion. Electrons generate free radicals in vinyl terminated monomers leading to double bond opening, polymerization and crosslinking. A balance of properties, especially for over-print materials, is attained by using oligomers which are terminated with acrylate functionality. Familiar ink and coating materials, such as polyesters, polyurethanes, epoxies and acrylates themselves are used in developing reactive oligomers which enhance the flexibility and other properties of the cured and crosslinked system. Since there are no extractable initiators used in EB curable inks and over-print systems, Sun Chemical has been able to develop over-print materials that are compliant with US Food & Drug Administration regulations for direct food contact. Use of such systems could replace film laminates used atop printed materials to prevent leaching of extractables for compliance with direct food contact regulations.

The major suppliers of monomers and oligomers for inks, coatings and adhesives have addressed issues of toxicity, Clean Air Act compliance, food contact and a host of other areas of concern in contemporary industry. These have been driving factors in changing the portfolio of materials available to formulators over the past several decades. The energy required to convert formulations of inks, coatings or adhesives using electron beams is significantly less than that to use alternative drying systems, even with so called “high solids” content products. Besides eliminating volatile organic emissions (VOCs), EB curing also lessens potential greenhouse gas emissions.

Low-energy EB processing is used in making laminates of thin films or thin film overlays. Higher energy EB or even X-rays can be used to set the adhesive bonds between thicker substrates. Materials with very different coefficients of thermal expansion can be bonded with EB curable adhesives without creating the interfacial strain generated when using thermal curing. Pressure sensitive adhesives (PSAs) are also cured using EB. Formulated PSAs based on natural rubber or similar diene polymers crosslink at high product through-put rates on EB treatment. Acrylic adhesives often based on butyl (C4) and 2-ethyl hexyl acrylate (C8) monomers and combinations thereof provide transparent adhesives. When using such monomers by themselves, attention must be given to dose-rate effects in order to avoid unwarranted chain termination, which would reduce the molecular weight of the cured material. The propagation step of such in-situ polymerization/crosslinking reactions can be extended by reducing the dose-rate and increasing the residence time under the EB unit.

### Hydrogels

Another technology based on liquids that are coated and then irradiated is the manufacture of hydrogels. Radiation crosslinked hydrogels are based mainly on polyethylene oxides (PEO) dissolved at relatively low concentrations in water, ˜4% to ˜10%. Modest radiation exposure is needed to form a gel, less than 10 kGy. Polyethylene glycols (PEG), polyvinyl alcohols (PVA) and polyvinylpyrrolidone (PVP) have also been used in these systems. PVP is also very radiation responsive. Because of the excellent radiation response of the polymers used in making hydrogels, low-current research type accelerators, as Van de Graaffs, can be used in their manufacture. Gels as thick as 2 mm are produced. These materials have found use as wound dressings and for burn treatment. Because of their biocompatibility, there is considerable activity in evaluating hydrogels as transdermal drug delivery systems and as drug delivery systems that can be inserted into the body. Polyethylene oxide (PEO) is very amenable to radiation crosslinking. Hydrogels are also being evaluated to provide compliant surfaces in prosthetic devices.

### Medical Device Sterilization and Marking

EB sterilization is also used for medical disposable items and non-disposable items, such as hip and other joint replacement parts. These implants are made from combinations of metals, ceramics and plastics that can be EB treated and marked with detectable markers for security, validation, authentication and tracking.

### Other Applications

Most of the accelerators used in industry are used to enhance the properties of plastics and elastomers or to convert polymeric precursors that are applied as liquids to yield cured inks, coatings and adhesives, and hydrogels. All require radiation crosslinking in order to form materials of commercial interest and value.

With EB or X-ray sterilization, plastic components ought to be radiation tolerant, not discoloring nor degrading under the exposures needed to eliminate bioburdens. Besides the major end-use applications highlighted above, there are others worth noting. These are proven and effective industrial EB processes but are limited by the size of a given market or by still developing commercial acceptance.

### PTFE Degradation

Polytetrafluoroethylene (PTFE) chain scissions upon exposure to electron beam irradiation. After reducing the molecular weight, this highly crystalline polymer can be ground into fine powders (2 μm to 20 μm). Small amounts of these powders (2% to 5%) are incorporated into printing inks and coatings to act as internal slip additives. This prevents very decorative printing from abrading other printed material packaged next to it in a container.

### Filter Membranes

Surface grafting is used to modify the hydrophilicity or hydrophobicity of filter membranes. Micro-porous polyvinylidene fluoride (PVdF) films are used. Graft monomers are selected based upon the desired end-product use.

### Semi-Conductor Treatment

Diodes and transistors are irradiated using EB to induce permanent or transitory modifications in the electrical properties of these devices. Absorbed doses around 100 kGy are used in these applications. Tests are based on the direct measurement of the current as a function of voltage before and after EB processing. Results show that the alterations in the drift speeds of the load carriers cause reductions of the reversal recovery times for these devices. Thus, semiconductors are more suitable for applications in high frequency and high power circuits.

### Gem Stone Irradiation

Electron beam irradiation will alter the color of some gem stones so as to enhance their commercial value. Topaz, rubelita, quartz, citrine, ametista and even diamonds have been irradiated for this effect. Since very high doses are often required, care is taken not to overheat the gems while they are being irradiated.

### Composite Curing

EB has been used to cure the matrix materials in carbon fiber composites. Recent studies have shown that such matrices can also be cured in a mold to produce shaped articles, such as vehicle fenders, using X-rays wherein the X-rays penetrate through molds and the product shape.

### Carbon Fiber Modification

Carbon fibers used in composite manufacture have been treated with EB in order to enhance the adhesion of the matrix to the fiber. Improvements in mechanical properties of cured composites were observed irrespective of the initial sizing on the fiber.

### Silicon-Carbide Fiber Manufacture

Silicon-carbide (SiC) fiber is made by first extruding polycarbosilane and then irradiating fiber strands with EB to crosslink the fiber. The conventional process involving heating the fiber produces a fiber with lower heat resistance due to the presence of oxygen. EB crosslinked SiC fiber can maintain high tensile strengthen up to 1700° C. while thermally crosslinked SiC maintain strength only up to 1200° C. This type of ceramic fiber is used in space applications.

### PTFE Crosslinking

PTFE can be crosslinked by electron beams at high temperatures (330-340° C.) while in an inert gas and slightly above its melt transition temperature. Increased mechanical properties and wear resistance make the crosslinked PTFE suitable for sliding parts, rollers and bearings. Commercial quantities of this material are being produced in Japan.

### Rubber Sheeting

Wide widths of calendared EB crosslinked sheeting are used for roofing and for pond and water retention basin linings and as material to prevent leakage from landfills. Sheeting is commonly made from EB crosslinkable polyolefins, notably formulated ethylene-propylene diene rubber (EPDM) which responds well to EB processing.

### Grafted Biologically Active Compounds

By incorporating enzymes into bio-compatible polyethylene glycols and then using EB to crosslink the polymer into a gel, the enzyme is immobilized and its lifetime and storage time are enhanced.

The examples disclosed herein are provided to illustrate the inventive concept and are not intended to be taken as limitation of the scope of the invention. The disclosures of the patents and non-patent references cited herein are hereby incorporated by reference in their entireties.

What is claimed is:

1. A composition comprising a detectable marker immobilized on a surface pre-treated with an electron beam.

2. The composition of claim 1, wherein the surface comprises an inorganic surface of a metal, a ceramic, a semi-conductor, a crystal or a gemstone.

3. The composition of claim 2, wherein the surface comprises a metal of an ingot, a bar, a shaped metal component, a beam, a wire, or a chain.

4. The composition of claim 1, wherein the surface comprises one or more polymers selected from the group consisting of a polyolefin, an halogenated polyolefin, polyethylene (PE), ethylene-propylene co-polymers (EPM), ethylene propylenediene elastomer copolymer (EPDM), polyvinyl chloride (PVC), ethylene vinylacetate (EVA), ethylene ethylacrylate (EEA), polyvinylidene fluoride (PVDF), ethylene tetrafluoroethylene (ETFE), tetraethyleneglycol dimethacrylate (TEGDM), trimethylol propane triacrylate (TMPTA), and triallyl cyanurate (AC).

5. The composition of claim 4, wherein the one or more polymers are chemically bonded to one or more of a stabilizer, a lubricant, a plasticizer, a fire retardant.

6. The composition of claim 4, wherein the one or more polymers are formed into a coating of a metal of an ingot, a bar, a shaped metal component, a beam, a wire, or a chain.

7. The composition of claim 1, wherein the surface is a surface of an item of jewelry.

8. The composition of claim 1, wherein the detectable marker is selected from the group consisting of an optical marker, a dye, a fluorophore, a biomolecule, a metal and a rare earth element.

9. The composition of claim 8, wherein the detectable marker is a biomolecule selected from the group consisting of nucleic acid, a protein, a peptide, a co-enzyme and a vitamin.

10. The composition of claim 1, wherein the detectable marker is chemically bonded to the electron beam-treated surface.

11. The composition of claim 10, wherein the detectable marker is covalently bonded to cross linked molecules of the electron beam-treated surface.

12. A method of binding a detectable marker to a surface, wherein the method comprises the steps of:
exposing the surface to an electron beam producing an electron beam-treated surface;
applying a detectable marker to the electron beam-treated surface; and
thereby producing a surface-bound detectable marker on the treated surface.

13. The method of claim 12, wherein the detectable marker is selected from the group consisting of an optical marker, a dye, a fluorophore, a biomolecule, a metal and a rare earth element.

14. The method of claim 13, wherein the detectable marker is a biomolecule selected from the group consisting of nucleic acid, a protein, a peptide, a co-enzyme and a vitamin.

15. The method of claim 13, wherein the detectable marker comprises one or more nucleic acid molecules.

16. The method of claim 14, further comprising:
extracting a sample of the detectable marker comprising one or more nucleic acid molecules from the electron beam-treated surface; and
identifying the extracted sample of nucleic acid molecules as a nucleic acid marker molecule.

17. The method of claim 16, wherein the identifying of the extracted nucleic acid sample is by amplifying the extracted nucleic acid using a polymerase chain reaction (PCR) to produce one or more specific length amplicons.

18. The method of claim 17, wherein the one or more specific length amplicons are subjected to capillary electrophoresis.

19. The method of claim 15, wherein the nucleic acid molecules applied to the treated surface are at a concentration of least about one femtogram per liter (˜10⁻¹⁵ g/L).

20. The method of claim 15, wherein the at least one of the nucleic acid molecules includes a marker nucleotide sequence.
